(19) United States
(12) Patent Application Publication
(10) Pub. No.: US 2023/0233591 A1
(43) Pub. Date: Jul. 27, 2023

(54) METHOD FOR TREATING POXVIRIDAE INFECTIONS

(71) Applicant: OYAGEN, INC., Rochester, NY (US)

(72) Inventors: Harold C. Smith, Rochester, NY (US); Ryan P. Bennett, Clifton Springs, NY (US)

(21) Appl. No.: 17/918,970

(22) PCT Filed: Apr. 14, 2021
(86) PCT No.: PCT/US2021/027354

§ 371 Date: Oct. 14, 2022

Related U.S. Application Data
(60) Provisional application No. 63/010,021, filed on Apr. 14, 2020.

(51) Int. Cl. A61K 31/7064; A61P 31/12

(52) U.S. Cl. CPC A61K 31/7064; A61P 31/12

(57) ABSTRACT

Disclosed herein are methods, compositions and kits for treating and inhibiting Poxviridae virus infections, for example, Vaccinia virus infections. Further disclosed are stop-gap methods for controlling the spread of Poxviridae virus infections.

### FIELD

Disclosed herein are methods, compositions and kits for treating and inhibiting Poxviridae virus infections and their associated diseases. In particular, disclosed herein are methods, compositions, and kits for treating and inhibiting, for example, Vaccinia virus infections. Further disclosed are stop-gap methods for controlling the spread of Poxviridae virus infections.

### SUMMARY OF THE DISCLOSURE

Various aspects of the present disclosure are described in the below Paragraphs 1-26 and in the noted combinations thereof, as follows:

Paragraph 1. A method for treating a subject infected with a Poxviridae virus, comprising: administering to a subject a therapeutically effective amount of 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula:

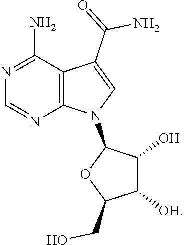 [SMILES: NC(=O)c1cn([C@@H]2O[C@H](CO)[C@@H](O)[C@H]2O)c2ncnc(N)c12]

Paragraph 2. A method for treating a subject infected with a Poxviridae virus, comprising: administering to a subject a therapeutically effective amount of 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula:

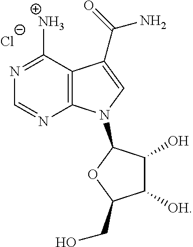 [SMILES: [Cl-] ; NC(=O)c1cn([C@@H]2O[C@H](CO)[C@@H](O)[C@H]2O)c2ncnc([NH3+])c12]

Paragraph 3. The method according to either Paragraph 1 or 2, wherein the effective amount is from about 0.1 mg/kg to about 5 mg/kg of the body mass of the subject.

Paragraph 4. The method according to either Paragraph 1 or 2, wherein the effective amount is selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 5.0 mg/kg of the body mass of the subject.

Paragraph 5. A method for inhibiting the RNA-dependent RNA polymerase of the Poxviridae virus, comprising administering to a subject having a Poxviridae virus infection a therapeutically effective amount of:
i) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula:

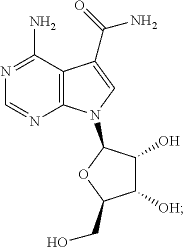 [SMILES: NC(=O)c1cn([C@@H]2O[C@H](CO)[C@@H](O)[C@H]2O)c2ncnc(N)c12]

ii) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula:

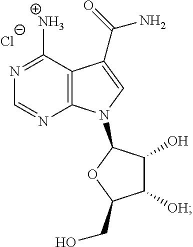 [SMILES: [Cl-] ; NC(=O)c1cn([C@@H]2O[C@H](CO)[C@@H](O)[C@H]2O)c2ncnc([NH3+])c12]

or
iii) mixtures thereof.

Paragraph 6. The method according to Paragraph 5, wherein the effective amount is from about 0.1 mg/kg to about 5 mg/kg of the body mass of the subject.

Paragraph 7. The method according to Paragraph 5, wherein the effective amount is selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 5.0 mg/kg of the body mass of the subject.

Paragraph 8. A stop-gap method for preventing the spread of Poxviridae virus comprising administering to a subject having a Poxviridae virus infection a therapeutically effective amount of:
i) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula:

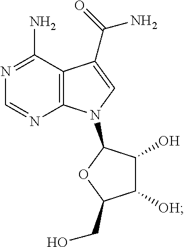 [SMILES: NC(=O)c1cn([C@@H]2O[C@H](CO)[C@@H](O)[C@H]2O)c2ncnc(N)c12]

ii) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula:

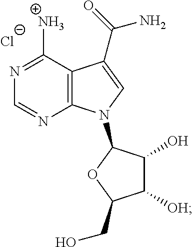 [SMILES: [Cl-] ; NC(=O)c1cn([C@@H]2O[C@H](CO)[C@@H](O)[C@H]2O)c2ncnc([NH3+])c12]

or
iii) mixtures thereof.

Paragraph 9. The method according to Paragraph 8, wherein the effective amount is from about 0.1 mg/kg to about 5 mg/kg of the body mass of the subject.

Paragraph 10. The method according to Paragraph 8, wherein the effective amount is selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 5.0 mg/kg of the body mass of the subject.

Paragraph 11. A method for preventing the emergence of a drug-resistant strain of a Poxviridae virus, comprising administering to a subject having a Poxviridae virus infection a therapeutically effective amount of:
i) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula:

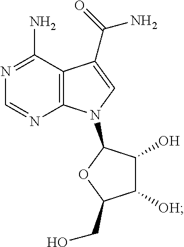 [SMILES: NC(=O)c1cn([C@@H]2O[C@H](CO)[C@@H](O)[C@H]2O)c2ncnc(N)c12]

ii) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula:

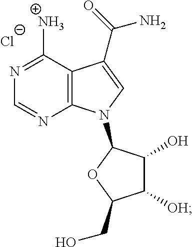 [SMILES: [Cl-] ; NC(=O)c1cn([C@@H]2O[C@H](CO)[C@@H](O)[C@H]2O)c2ncnc([NH3+])c12]

or
iii) mixtures thereof.

Paragraph 12. The method according to Paragraph 11, wherein the effective amount is from about 0.1 mg/kg to about 5 mg/kg of the body mass of the subject.

Paragraph 13. The method according to Paragraph 11, wherein the effective amount is selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 5.0 mg/kg of the body mass of the subject.

Paragraph 14. A method of prophylactically treating a subject uninfected with an Poxviridae virus comprising administering to an uninfected subject reasonably suspected as having been exposed, of currently being exposed, or in the future of being exposed to an Poxviridae virus a therapeutically effective amount of:
i) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula:

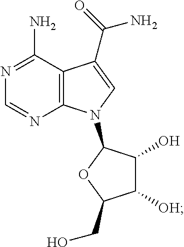 [SMILES: NC(=O)c1cn([C@@H]2O[C@H](CO)[C@@H](O)[C@H]2O)c2ncnc(N)c12]

ii) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula:

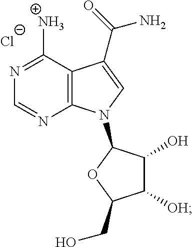 [SMILES: [Cl-] ; NC(=O)c1cn([C@@H]2O[C@H](CO)[C@@H](O)[C@H]2O)c2ncnc([NH3+])c12]

or
iii) mixtures thereof.

Paragraph 15. The method according to Paragraph 14, wherein the effective amount is from about 0.1 mg/kg to about 5 mg/kg of the body mass of the subject.

Paragraph 16. The method according to Paragraph 14, wherein the effective amount is selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 5.0 mg/kg of the body mass of the subject.

Paragraph 17. A pharmaceutical composition comprising:

a) a therapeutically effective amount of:
i) 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide having the formula:

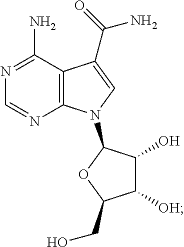 [SMILES: NC(=O)c1cn([C@@H]2O[C@H](CO)[C@@H](O)[C@H]2O)c2ncnc(N)c12]

ii) 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide hydrochloride having the formula:

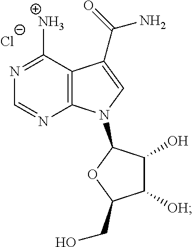 [SMILES: [Cl-] ; NC(=O)c1cn([C@@H]2O[C@H](CO)[C@@H](O)[C@H]2O)c2ncnc([NH3+])c12]

or
iii) mixtures thereof; and

b) the balance a pharmaceutically acceptable carrier.

Paragraph 18. The composition according to Paragraph 17, comprising from about 10 mg to about 250 mg of 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide and/or the hydrochloride salt thereof.

Paragraph 19. The composition according to either Paragraph 17 or 18, wherein the composition is in the form of an oral-use composition.

Paragraph 20. The composition according to either Paragraph 17 or 18, wherein the composition is in the form of a pill.

Paragraph 21. The composition according to either Paragraph 17 or 18, wherein the composition is in the form of a capsule.

Paragraph 22. The composition according to either Paragraph 17 or 18, wherein the composition is in the form of a nasal delivery composition.

Paragraph 23. The composition according to either Paragraph 17 or 18, wherein the composition is in the form of a sterile injectable composition.

Paragraph 24. Use of a pharmaceutical composition according to either Paragraph 17 or 18 for the treatment of Poxviridae virus in combination with a vaccine, a therapeutic, and/or other small molecule drug intended for treatment of Coronavirus as a combination therapy to treat Poxviridae virus in a subject.

Paragraph 25. The use according to Paragraph 24, wherein the combination therapy is effective to inhibit and/or prevent the emergence of drug resistant strains of Poxviridae virus.

Paragraph 26. A kit comprising a pharmaceutical composition, the composition comprising:

a) one or more Poxviridae virus inhibitors chosen from:
i) 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide having the formula:

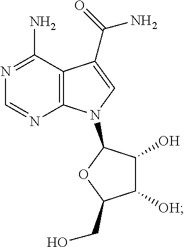 [SMILES: NC(=O)c1cn([C@@H]2O[C@H](CO)[C@@H](O)[C@H]2O)c2ncnc(N)c12]

ii) 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide hydrochloride having the formula:

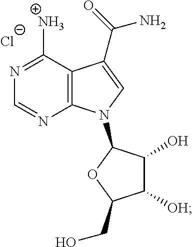 [SMILES: [Cl-] ; NC(=O)c1cn([C@@H]2O[C@H](CO)[C@@H](O)[C@H]2O)c2ncnc([NH3+])c12]

or
iii) mixtures thereof; and

b) optionally the balance a pharmaceutically acceptable carrier.

These and other objects, features, and advantages of this disclosure will become apparent from the following detailed description of the various aspects of the disclosure taken in conjunction with the accompanying drawings.

### BRIEF DESCRIPTION OF THE FIGURES

FIG. 1 depicts the antiviral activity of the disclosed compound Oya1 on CPXV virus infected Huh7 cells at a MOI of 0.1 for 24 hours. The percent inhibition that the test compounds displayed is indicated by (●), whereas the percent toxicity is indicated by (▪).

FIG. 2 depicts the antiviral activity of the disclosed compound Oya1 on CPXV virus infected NR596 (VeroE6) cells at a MOI of 0.1 for 24 hours. The percent inhibition that the test compounds displayed is indicated by (●), whereas the percent toxicity is indicated by (▪).

FIG. 3 depicts the antiviral activity of the disclosed compound Oya2 on CPXV virus infected Huh7 cells at a MOI of 0.1 for 24 hours. The percent inhibition that the test compounds displayed is indicated by (●), whereas the percent toxicity is indicated by (▪).

FIG. 4 depicts the antiviral activity of the disclosed compound Oya2 on CPXV virus infected NR596 (VeroE6) cells at a MOI of 0.1 for 24 hours. The percent inhibition that the test compounds displayed is indicated by (●), whereas the percent toxicity is indicated by (▪).

FIG. 5 depicts the antiviral activity of the disclosed compound Oya1 on VACV virus infected Huh7 cells at a MOI of 0.1 for 24 hours. The percent inhibition that the test compounds displayed is indicated by (●), whereas the percent toxicity is indicated by (▪).

FIG. 6 depicts the antiviral activity of the disclosed compound Oya1 on VACV virus infected NR596 (VeroE6) cells at a MOI of 0.1 for 24 hours. The percent inhibition that the test compounds displayed is indicated by (●), whereas the percent toxicity is indicated by (▪).

FIG. 7 depicts the antiviral activity of the disclosed compound Oya2 on VACV virus infected Huh7 cells at a MOI of 0.1 for 24 hours. The percent inhibition that the test compounds displayed is indicated by (●), whereas the percent toxicity is indicated by (▪).

FIG. 8 depicts the antiviral activity of the disclosed compound Oya2 on VACV virus infected NR596 (VeroE6) cells at a MOI of 0.1 for 24 hours. The percent inhibition that the test compounds displayed is indicated by (●), whereas the percent toxicity is indicated by (▪).

### DETAILED DESCRIPTION OF THE DISCLOSURE

The materials, compounds, compositions, articles, and methods described herein may be understood more readily by reference to the following detailed description of specific aspects of the disclosed subject matter and the Examples included therein.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

All percentages, ratios and proportions herein are by weight, unless otherwise specified. All temperatures are in degrees Celsius (° C.) unless otherwise specified.

The terms “a” and “an” are defined as one or more unless this disclosure explicitly requires otherwise.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Any embodiment of any of the disclosed methods or compositions can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Any embodiment of any of the disclosed compounds or methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

As used herein the term Poxviridae refers to the poxvirus family. Four genera of poxviruses may infect humans: orthopoxvirus, parapoxvirus, yatapoxvirus, molluscipoxvirus. Orthopox: smallpox virus (variola), vaccinia virus, cowpox virus, monkeypox virus; Parapox: orf virus, pseudocowpox, bovine papular stomatitis virus; Yatapox: tanapox virus, yaba monkey tumor virus; Molluscipox: molluscum contagiosum virus (MCV). The most common are vaccinia (seen on Indian subcontinent) and molluscum contagiosum, but monkeypox infections are rising (seen in west and central African rainforest countries).

As used herein, the term “Vaccinia” refers to a family of large, complex, enveloped virus belonging to the poxvirus family. It is characterized by having a linear double-stranded CAN genome approximately 190 kbp in length, which encodes approximately 250 genes. The dimensions of the virion are roughly 360×270×250 nm, with a mass of about 5-10 fg. Included with in the genus are the species buffalo virus, cantagalo virus, rabbitpox virus Utrecht, Vaccinia virus Ankara, Vaccinia virus Copenhagen and Vaccinia virus WR.

As used herein, the term “subject” refers to a human or an animal. The term subject can refer to a human or animal exposed to or infected with a virus of the Poxviridae family. More particularly, the term subject can refer to a human or animal that has been diagnosed with a Vaccinia virus infection. The term subject also includes humans or animals that have been exposed to a Poxviridae virus, and more particularly the Vaccinia virus, but are not symptomatic.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like are encompassed within the term “treating,” and refer to reducing the probability of developing a disorder or condition in a subject, who does not have, but is at risk of or susceptible to developing a disorder or condition.

As used herein, “pharmaceutically acceptable” means physiologically tolerable, for either human or veterinary applications. In addition, “pharmaceutically acceptable” is meant for a material that is not biologically or otherwise undesirable, i.e., the material may be administered to a subject without causing any undesirable biological effects or interacting in a deleterious manner with any of the other components of the pharmaceutical composition in which it is contained. Essentially, the pharmaceutically acceptable material is nontoxic to the recipient. The carrier would naturally be selected to minimize any degradation of the active ingredient, to minimize any adverse side effects in the subject, and to optimize formulation for drug delivery and dosing to the target tissues infected by Poxviridae as would be well known to one of skill in the art. For a discussion of pharmaceutically acceptable carriers and other components of pharmaceutical compositions, see, e.g., Remington's Pharmaceutical Sciences, 18th ed., Mack Publishing Company, 1990.

“Test agents” or otherwise “test compounds” as used herein refers to an agent or compound that is to be screened in one or more of the assays described herein. Test agents include compounds of a variety of general types including, but not limited to, small organic molecules, known pharmaceuticals, polypeptides; carbohydrates such as oligosaccharides and polysaccharides; polynucleotides; lipids or phospholipids; fatty acids; steroids; or amino acid analogs. Test agents can be obtained from libraries, such as natural product libraries and combinatorial libraries. In addition, methods of automating assays are known that permit screening of several thousands of compounds in a short period.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and describe the methods and/or materials in connection with which the publications are cited.

As used herein “stop-gap” refers to the administration of the disclosed compounds to ameliorate the spread of a coronavirus and emergence of drug resistant strains. A stop-gap administration is a temporary measure designed to control the spread of the virus until medical personnel can evaluate the extent of infection and/or the source.

Smallpox was the first disease to be widely prevented by vaccination, due to pioneering work by the English physician and scientist Edward Jenner, in the eighteenth century, using cowpox virus. Vaccinia virus is the active constituent of the vaccine that eradicated smallpox, making it the first human disease to be eradicated. This endeavor was carried out by the World Health Organization under the Smallpox Eradication Program.

Details associated with the embodiments described above and others are described below.

### Methods

Disclosed herein are methods for treating a subject having a viral infection caused by a virus of the family Poxviridae, particularly a coronavirus infection. Although a particular method may be described herein as an antiviral treatment or prophylactic against a specific Poxviridae virus (e.g., the Vaccinia virus), any such method is meant to also include an antiviral treatment or prophylactic against other viruses in the Poxviridae family.

Compounds disclosed herein for treating a coronavirus infection have Formula I.

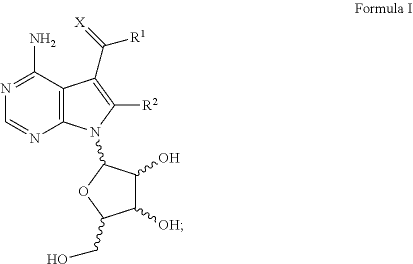

wherein X is chosen from O or S;
R¹ is chosen from —NH₂, —NHOH and —NHNH₂; and
R² is chosen from hydrogen and —NHNH₂; or
a pharmaceutically acceptable salt thereof.

As used herein, a compound can include, without limitation, derivatives, homologs, analogs, metabolites, prodrugs, conjugates, complexes, salts, free acids, bases, solvates, enantiomers, isomers, hydrates, esters, racemates, and/or polymorphs of the compounds described herein (including, without limitation, the compounds identified herein as “Oya1” and “Oya2”), and/or any formulations thereof. In certain embodiments, the term derivatives can refer to any composition that is derived from the scaffold of the compound using chemical reactions on the compound or using de nova whole molecule chemical synthesis.

The disclosed compounds can be used to treat, cure, abate, minimize, control, and/or lessen the effects of a virus of the family Poxviridae in humans and animals and spread through communities within and distal to the outbreak zone. The disclosed compounds can also be used to slow the rate of coronavirus spread in a population. The disclosed compounds can also be used to prevent or reduce the emergence of drug-resistant strains of coronaviruses by preventing coronavirus spread in a population. The disclosed compounds when administered to a subject in need of treatment can be used to stop the spread of a coronavirus virus infection. As such, the compounds disclosed herein can be administered as part of a combination therapy with one or more drugs or other pharmaceutical agents.

Four genera of poxviruses may infect humans: orthopoxvirus, parapoxvirus, yatapoxvirus, molluscipoxvirus. Orthopox: smallpox virus (variola), vaccinia virus, cowpox virus, monkeypox virus; Parapox: orf virus, pseudocowpox, bovine papular stomatitis virus; Yatapox: tanapox virus, yaba monkey tumor virus; Molluscipox: molluscum contagiosum virus (MCV). The most common are vaccinia (seen on Indian subcontinent) and molluscum contagiosum, but monkeypox infections are rising (seen in west and central African rainforest countries).

Non-limiting examples of Poxviridae viruses include the genera include Orthopox, Parapox, Yatapox and Molluscipox. As indicated in Formula I all enantiomers and diasteriomers of Formula I are included as compounds suitable for use in the herein disclosed methods for treating a subject infected with a virus of the family Poxviridae.

Further disclosed herein is the use of the disclosed compounds for making a medicament useful in treating a subject infected with one or more viruses of the family Poxviridae. The medicament can comprise one or more of the compounds having Formula I.

One aspect of the disclosure relates to methods for treating a subject having a Poxviridae virus infection, comprising contacting the subject with a therapeutically effective amount of one or more compounds of Formula II:

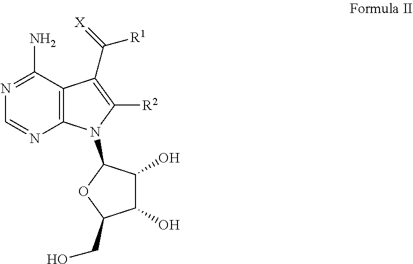

wherein X, R¹ and R² are the same as disclosed herein above; or
a pharmaceutically acceptable salt thereof.

One embodiment of this aspect comprises, contacting a subject having an Poxviridae virus infection with a therapeutically effective amount of 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide (also known as sangivamycin CAS No. 18417-89-6) and designated herein as “Oya1” having Formula IIa:

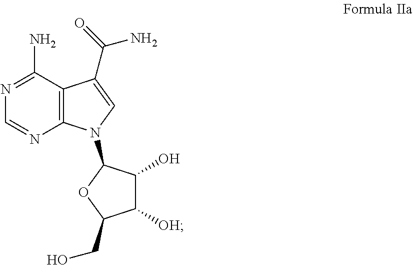 [SMILES: NC(=O)c1cn([C@@H]2O[C@H](CO)[C@@H](O)[C@H]2O)c2ncnc(N)c12]

This compound is available from Sigma-Aldrich™.

A further embodiment of this aspect comprises, contacting a subject having an Poxviridae virus infection with a therapeutically effective amount of 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride (“Oya1 hydrochloride,” also known as sangivamycin hydrochloride CAS No. 21090-35-7) and designated herein as “Oya2” having Formula Ib:

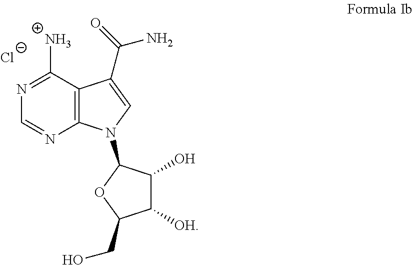 [SMILES: [Cl-] ; NC(=O)c1cn([C@@H]2O[C@H](CO)[C@@H](O)[C@H]2O)c2ncnc([NH3+])c12]

A still further example of this embodiment comprises, contacting a subject having an Poxviridae virus infection with a therapeutically effective amount of 4-amino-7-((2R,3R,4S,5R)-3,4-dihydroxy-5-(hydroxymethyl)tetrahydrofuran-2-yl)-6-hydrazinyl-N-hydroxy-7H-pyrrolo[2,3-d]pyrimidine-5-carboximidamide having Formula III:

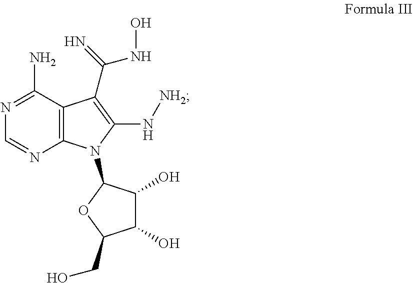 [SMILES: N=C(NO)c1c(NN)n([C@@H]2O[C@H](CO)[C@@H](O)[C@H]2O)c2ncnc(N)c12]

or a pharmaceutically acceptable salt thereof. This compound is referred to as ‘Oya1-Like Molecule 5 (SLM5) and is available from the NIH Developmental Therapeutics Program (DTP).

A still further embodiment comprises, contacting a subject having an Poxviridae virus infection with a therapeutically effective amount of 4-amino-7-((2R,3R,4S,5R)-3,4-dihydroxy-5-(hydroxymethyl)tetrahydrofuran-2-yl)-6-hydrazinyl-7H-pyrrolo[2,3-d]pyrimidine-5-carboximidhydrazide having Formula IV:

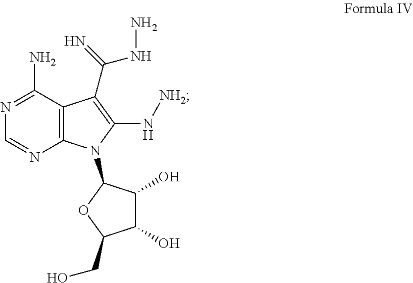 [SMILES: N=C(NN)c1c(NN)n([C@@H]2O[C@H](CO)[C@@H](O)[C@H]2O)c2ncnc(N)c12]

or a pharmaceutically acceptable salt thereof. This compound is referred to as ‘Oya1-Like Molecule 6 (SLM6) and is available from the NIH Developmental Therapeutics Program (DTP).

A another further embodiment comprises, contacting a subject having an Poxviridae virus infection with a therapeutically effective amount of 4-amino-7-((2R,3R,4S,5R)-3,4-dihydroxy-5-(hydroxymethyl)tetrahydrofuran-2-yl)-6-hydrazinyl-7H-pyrrolo[2,3-d]pyrimidine-5-carboximidamide having Formula V:

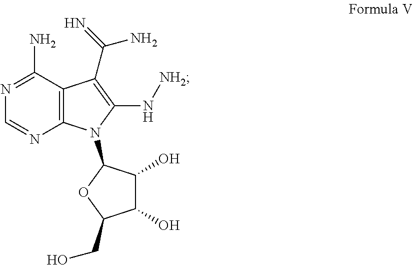 [SMILES: N=C(N)c1c(NN)n([C@@H]2O[C@H](CO)[C@@H](O)[C@H]2O)c2ncnc(N)c12]

or a pharmaceutically acceptable salt thereof. This compound is referred to as ‘Oya1-Like Molecule 7 (SLM7) and is available from the NIH Developmental Therapeutics Program (DTP).

A yet further embodiment comprises, contacting a subject having an Poxviridae virus infection with a therapeutically effective amount of 4-amino-7-((2R,3R,4S,5R)-3,4-dihydroxy-5-(hydroxymethyl)tetrahydrofuran-2-yl)-7H-pyrrolo[2,3-d]pyrimidine-5-carbothioamide (thioOya1) having Formula VI.

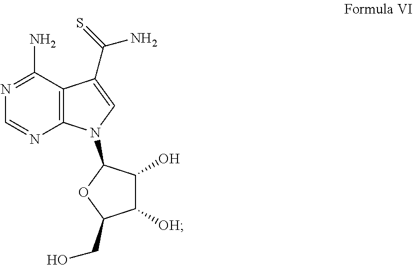 [SMILES: NC(=S)c1cn([C@@H]2O[C@H](CO)[C@@H](O)[C@H]2O)c2ncnc(N)c12]

or a pharmaceutically acceptable salt thereof.

### Combination Therapy

One aspect of the disclosure relates to the use of a pharmaceutical composition as disclosed herein for the treatment of Poxviridae virus in combination with a vaccine, a therapeutic, and/or other small molecule drug intended for treatment of Poxviridae virus as a combination therapy to treat Poxviridae virus in a subject. One embodiment of this aspect involves using the combination therapy in a manner and under conditions effective to inhibit and/or prevent the emergence of drug resistant strains of Poxviridae virus.

### Polymerase Inhibition

One aspect disclosed herein are methods for treating a subject infected with the Poxviridae virus, comprising: administering to a subject a therapeutically effective amount of 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula:

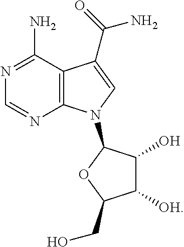 [SMILES: NC(=O)c1cn([C@@H]2O[C@H](CO)[C@@H](O)[C@H]2O)c2ncnc(N)c12]

Further disclosed herein are methods for treating a subject infected with the Poxviridae virus, comprising: administering to a subject a therapeutically effective amount of 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula:

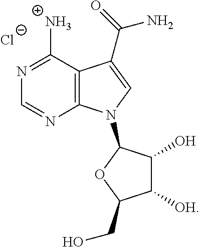 [SMILES: [Cl-] ; NC(=O)c1cn([C@@H]2O[C@H](CO)[C@@H](O)[C@H]2O)c2ncnc([NH3+])c12]

Still further disclosed is a method for inhibiting the polymerase of the poxvirus, comprising administering to a subject having a Poxviridae virus infection a therapeutically effective amount of:
i) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula:

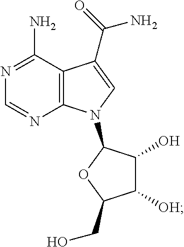 [SMILES: NC(=O)c1cn([C@@H]2O[C@H](CO)[C@@H](O)[C@H]2O)c2ncnc(N)c12]

ii) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula:

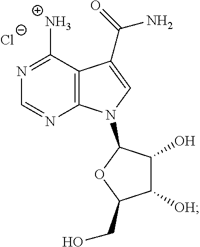 [SMILES: [Cl-] ; NC(=O)c1cn([C@@H]2O[C@H](CO)[C@@H](O)[C@H]2O)c2ncnc([NH3+])c12]

or
iii) mixtures thereof.

The disclosed methods provide a single dose of Oya1 or Oya2 based upon the body mass of the subject being treated. Therefore, a single dose of Oya1 or Oya2 can range from about 0.1 mg/kg to about 5 mg/kg of the subject's body mass.

For example, the dose can comprise any amount from about 0.1 mg/kg to about 5 mg/kg of the body mass of the subject being treated.

For example, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 5.0 mg/kg of body mass.

Further disclosed is a “stop-gap” method for controlling the spread of the poxvirus outbreak and the spread within an affected population and from people travelling from the outbreak zone or travelling into and around or nearby the outbreak zone. What is meant herein by “stop-gap” is a method for temporarily halting the transmission of the Poxviridae virus among a population group until such time when viral strain-specific therapies become available whether those are small molecule, vaccine, or other antiviral therapeutics. It has been determined that transmission of the virus can occur by close contact with an infected subject, wherein transmission is due in part to contact with a cough aspirant or other bodily fluid. Without wishing to be limited by theory Oya1 or Oya2 is effective for eight days following a single maximum tolerated dose or with repeated daily submaximal doses in inhibiting the activity of coronavirus polymerase. Therefore, removing a subject from an area wherein infection has been found, but wherein the subject is otherwise asymptomatic, provides a means to protect the subject from infection, and protecting healthcare or other professional who must come in contact with the patient from disease transmission. Treatment of patients with or without symptoms, at large or in quarantine with Oya1 or Oya2 will reduced the replication of the virus and thereby mitigate the severity of and development of disease symptom, reduce the transmission the virus as a control measure and save lives for those who may be immunocompromised and at risk for sever complications and death. Thus, in one sense, a stop-gap in accordance with the present disclosure is a therapeutic treatment used to slow the progression of a disease within an infected patient or to reduce the spread of a viral infection to uninfected people within an outbreak zone as a temporary means of controlling morbidity and mortality.

Therefore, disclosed herein is a method of preventing the transmission and spread of Poxviridae virus, comprising removing a subject from a site of infection risk and administering an effective amount of Oya1 or Oya2 to the subject. The subject once treated, can be isolated for the purposes of observation. After 8 days, an additional bolus of Oya1 or Oya2 can be further administered if warranted.

Further disclosed herein is a method of prophylactically treating a subject uninfected with an Poxviridae virus, but reasonably suspected as having been exposed, of currently being exposed, or in the future of being exposed to an Poxviridae virus, by administering a therapeutically effective amount of Oya1 or Oya2 to the uninfected subject. This method is intended to prevent and/or reduce new infections of Poxviridae virus.

Further disclosed herein is a method of treating people prophylactically who have not been infected but need protection from infection when entering outbreak zones, treating infected individuals, decontaminating infected areas, or participating in maintaining civil rule of law by administering an effective amount of Oya1 or Oya2 to uninfected subjects a bolus of Oya1 or Oya2 prior to and during contact with infected people or surfaces.

Disclosed herein is the use of Oya1 and/or Oya2 to treat a Poxviridae virus infection. Further disclosed is the use of Oya1 and Oya2 to treat one or more of the virus infections chosen from genera Orthopox, Parapox, Yatapox and Molluscipox.

### Compositions

Disclosed herein are pharmaceutical compositions for use in treating a subject infected with a Poxviridae virus, the pharmaceutical compositions comprising:

a) a therapeutically effective amount of:
i) 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide having the formula:

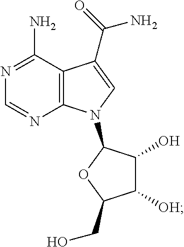 [SMILES: NC(=O)c1cn([C@@H]2O[C@H](CO)[C@@H](O)[C@H]2O)c2ncnc(N)c12]

ii) 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide hydrochloride having the formula:

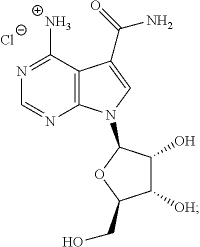 [SMILES: [Cl-] ; NC(=O)c1cn([C@@H]2O[C@H](CO)[C@@H](O)[C@H]2O)c2ncnc([NH3+])c12]

or
iii) mixtures thereof; and

b) the balance a pharmaceutically acceptable ingredients.

In one embodiment the therapeutically effective amount is from about 1 mg/kg to about 3 mg/kg of the body mass of the subject to be treated.

The disclosed compositions can comprise from about 10 mg to about 250 mg. In one aspect the disclosed single dose compositions of Oya1 or Oya2 can comprise any amount from about 10 mg to about 250 mg. For example, the disclosed compositions can comprise 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 90 mg, 91 mg, 92 mg, 93 mg, 94 mg, 95 mg, 96 mg, 97 mg, 98 mg, 99 mg, 100 mg, 101 mg, 102, mg, 103, mg, 104 mg, 105 mg, 106 mg, 107 mg, 108 mg, 109 mg, 120 mg, 121 mg, 122 mg, 123 mg, 124 mg, 125 mg, 126 mg, 127 mg, 128 mg, 129 mg, 130 mg 31 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 190 mg, 191 mg, 192 mg, 193 mg, 194 mg, 195 mg, 196 mg, 197 mg, 198 mg, 199 mg, 200 mg, 201 mg, 202, mg, 203, mg, 204 mg, 205 mg, 206 mg, 207 mg, 208 mg, 209 mg, 210 mg, 212 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg of Oya1 and/or Oya2.

In general, the disclosed pharmaceutical compositions include but are not limited to those suitable for oral, rectal, nasal, topical (including buccal and sub-lingual), transdermal, vaginal or parenteral (including intramuscular, subcutaneous and intravenous) administration or in a form suitable for administration by inhalation or insufflation. The compositions can, where appropriate, be conveniently presented in discrete dosage units and may be prepared by any of the methods well known in the art of pharmacy. Pharmaceutical compositions suitable for oral administration are conveniently presented as discrete units such as capsules, cachets or tablets, each containing a predetermined amount of the active substance.

### Oral-Use Compositions

Disclosed herein are compositions for oral delivery of 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide (Oya1) and/or 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide hydrochloride (Oya2). The compositions comprise:
a) from about 10 mg to about 250 mg by weight of Oya1, Oya2 or mixtures thereof; and
b) a pharmaceutically acceptable ingredients.

The disclosed oral use compositions can be in the form of a liquid composition or a solid in the form of a tablet or flowable powder. The disclosed pharmaceutically-acceptable excipients include, but are not limited to, polymers, resins, plasticizers, fillers, lubricants, solvents, co-solvents, surfactants, preservatives, sweetener agents, flavoring agents, buffer systems, pharmaceutical-grade dyes or pigments, and viscosity agents.

When present the coating can contain a plasticizer and possibly other coating excipients such as coloring agents, talc, and/or magnesium stearate, many of which are well known in the coating art. In particular, anionic carboxylic acrylic polymers usually can contain 10-25% by weight of a plasticizer, especially dibutyl phthalate, polyethylene glycol, triethyl citrate and triacetin. Conventional coating techniques such as spray or pan coating are employed to apply the coating. As previously mentioned, the coating thickness must be sufficient to ensure that the oral dosage form remains intact until the desired site of topical delivery in the lower intestinal tract is reached.

Liquid compositions comprise a suitable liquid carrier, for example, sterilized water. In addition, the liquid compositions can comprise buffers, preservatives, flavoring agents and co-solvents.

### Nasal Delivery Compositions

Disclosed herein are compositions for delivery of the disclosed compounds via nasal inhalation. The compositions which are inhaled can reside in the nostril or sinus cavities or can percolate downward and flow into the lungs. Without wishing to be limited by theory it is believed there is a higher concentration of Poxviridae virus in the nose and nasal passages. The compositions for nasal delivery are fine powders or nebulized solutions comprising Oya1 and/or Oya2 in an amount from about 10 mg to about 250 mg per inhaled dose.

### Sterile Injectable Compositions

### Administration of Sterile Injectable Composition

A disclosed sterile injectable composition can be injected using any of the known methods in the art. Particularly, a composition can be administered by means of an injection device suitable for intraepidermal and/or intradermally and/or subcutaneously.

The injection device particularly when a disclosed composition is administered by means of an injection device suitable for intraepidermal and/or intradermally and/or subcutaneously, can be selected from a syringe, a set of microsyringes, a laser device, a hydraulic device, an injection gun, a needleless injection device, or a rolling with microneedles. In one embodiment the injection device is chosen from a syringe or a set of microsyringe.

In a further embodiment, the injection device can be adapted to the technique of mesotherapy. Mesotherapy is a treatment technique by intraepidermal and/or intradermally and/or subcutaneously active(s) product(s). The administration intraepidermal and/or intradermally and/or subcutaneously according to the present disclosure is to inject a disclosed composition in an epidermal region, dermo-epidermal and/or dermal.

In addition, the injection device can comprise any conventionally used injection such as hypodermic needle or cannula. For example, a needle or a cannula according to the present disclosure can have a diameter ranging between 18 and 34 G. In one embodiment the diameter can be from about 25 to about 32 G. The length can vary from about 4 to about 70 mm. In one embodiment the diameter is from about 4 to about 25 mm. The needles used to inject the disclosed sterile compositions can be disposable. Advantageously, the needle or cannula is associated with a syringe or other device capable of delivering through the needle or cannula disclosed injectable composition.

According to one embodiment, a catheter may be inserted between the needle/cannula and syringe. In known manner, the syringe can be operated manually by the practitioner or by a syringe holder as guns.

The disclosed injectable sterile compositions comprise:
a) from about 10 mg to about 250 mg of a compound chosen from 4-amino-7-((2R,3R,4S,5R)-3,4-dihydroxy-5-(hydroxymethyl)-tetrahydrofuran-2-yl)-7H-pyrrolo[2,3-d]pyrimidine-5-carboxamide and/or the hydrochloride salt thereof (Oya1 and/or Oya2); and
b) a pharmaceutically acceptable carrier.

In one embodiment the pharmaceutically acceptable carrier is deionized, sterile water.

The injectable compositions can further comprise one or more isotonic agents suitable for the preparation of a disclosed composition, for example a sugar and/or sodium chloride. The acceptable carrier can be a balanced salt solution, for example, phosphate buffered saline.

In addition, the composition can further comprise an antioxidant, for example, glutathione, ellagic acid, spermine, resveratrol, retinol, L-carnitine, polyols, polyphenols, flavonols, theaflavins, catechins, caffeine, ubiquinol, ubiquinone, and mixture thereof.

In a further embodiment the disclosed composition can further comprise any excipient commonly used in the technical field, for example, mono- and/or di-hydrated dihydrogenophosphate sodium and sodium chloride, in physiological concentrations. The amounts of additional active agents and/or excipients of course depend on the nature of the desired properties determined by the formulator, the desired effect, and the destination of the composition according to the invention.

The disclosed composition, once prepared, can be sterilized by heat and directly packaged in suitable containers known in the art.

### Capsules

Disclosed herein is a pharmaceutical preparation comprising a capsule containing from about 10 mg to about 250 mg of 4-amino-7-((2R,3R,4S,5R)-3,4-dihydroxy-5-(hydroxymethyl)-tetrahydrofuran-2-yl)-7H-pyrrolo[2,3-d]pyrimidine-5-carboxamide and/or the hydrochloride salt thereof (Oya1 and/or Oya2).

The compositions in the form of a capsule comprise:
a) from about 10 mg to about 250 mg of a compound chosen from 4-amino-7-((2R,3R,4S,5R)-3,4-dihydroxy-5-(hydroxymethyl)-tetrahydrofuran-2-yl)-7H-pyrrolo[2,3-d]pyrimidine-5-carboxamide and/or the hydrochloride salt thereof (Oya1 and/or Oya2); and
b) a pharmaceutically acceptable carrier.

Another embodiment of the disclosed compositions are compositions in the form of a tablet. The tablets can comprise the same amount of Oya1 and/or Oya2 as the capsules. A further embodiment of the disclosed compositions are compositions in the form of a sterile injectable formulation. The injectable formulations can comprise the same amount of Oya1 and/or Oya2 as the capsules.

The disclosed compositions can comprise one or more pharmaceutically acceptable excipients, carriers or binders.

### Antiviral Control and Efficacy Testing

### CPXV Assay MOI 0.1

Oya1 and Oya2 activity was tested in a drug screen assay against CPXV at MOI 0.1 in Huh 7 and NR-596 (Vero E6) cells. Cyto-a-D-arab was used as the positive control. The cells were fixed at 24 hours.

Stock solutions of 100 mM Cyto-a-D-arab, 20 mM Oya1 and 20 mM Oya2 in DMSO. The following 96 Well plates were prepared in preparation for duplicate experiments:

TABLE 1

| Plate type | Cell type | Cell Seeding | Notes |
|---|---|---|---|
| 96 Well Operetta x2 | Vero E6 | 30,000 cells/well | Plate all wells |
| 96 Well Black Opaque | Vero E6 | 30,000 cells/well | No cells in 12^{th} |
| | | | col. |
| 96 Well Operetta x2 | Vero E6 | 30,000 cells/well | Plate all wells |
| 96 Well Black Opaque | Vero E6 | 30,000 cells/well | No cells in 12^{th} |
| | | | col. |

The plates were prepared in DMEM w/10% FBS. As indicated below the following serial dilutions were used in triplicate of each MOI.

TABLE 2

| Cyto-a-D-arab (μM) | | | Oya1 (μM) | | | Oya2 (μM) | | |
|---|---|---|---|---|---|---|---|---|
| 60.00 | 60.00 | 60.00 | 0.60 | 0.60 | 0.60 | 0.60 | 0.60 | 0.60 |
| 30.00 | 30.00 | 30.00 | 0.30 | 0.30 | 0.30 | 0.30 | 0.30 | 0.30 |
| 15.00 | 15.00 | 15.00 | 0.15 | 0.15 | 0.15 | 0.15 | 0.15 | 0.15 |
| 7.50 | 7.50 | 7.50 | 0.08 | 0.08 | 0.08 | 0.08 | 0.08 | 0.08 |
| 3.75 | 3.75 | 3.75 | 0.04 | 0.04 | 0.04 | 0.04 | 0.04 | 0.04 |
| 1.88 | 1.88 | 1.88 | 0.02 | 0.02 | 0.02 | 0.02 | 0.02 | 0.02 |
| 0.94 | 0.94 | 0.94 | 0.01 | 0.01 | 0.01 | 0.01 | 0.01 | 0.01 |
| 0.47 | 0.47 | 0.47 | 0.005 | 0.005 | 0.005 | 0.005 | 0.005 | 0.005 |

As seen in Table 2 all experiments were run in triplicate. 4 plates were run for efficacy (2×Huh 7, 2×Huh 7) and 2 plates for toxicity (1×Huh7, 1×Huh7) and 4 plates were run for efficacy (2×NR-596, 2×NR-596) and 2 plates for toxicity (1×NR-596, 1×NR-596).

50 μL of the compound solution to be tested dissolved in DMEM/10% FBS (Table 3) is pipetted to the corresponding wells of the efficacy and cytotoxicity plates. Prior to adding the test solution to the cell plates, Rainin liquidator-96 was used to mix all wells of the compound plate, by pipetting up and down 3 times with a maximum volume of 200 μL. All tips used in this step were discarded and fresh tips were used for the subsequent step.

50 μL of DMEM/10% FBS is added to the wells of the cytotoxicity plates to compensate for these plates not receiving any virus. 50 μL of DMEM/10% FBS is added to the 12^{th} column of all the efficacy plates to compensate for these wells not receiving virus later in the assay.

Huh-7 cells were plated at 30,000/well in black opaque or clear bottom 96-well Operetta plates one day prior assay. Cyto-a-D-arab, Oya1 and Oya2 were tested in an 8-point dose response curve using the serial dilutions of Table 3. Each dose was run in triplicate.

The results of this experiment are summarized in Table 3.

TABLE 3

| Drug | Virus | Cell type | CC₅₀ | IC₅₀ | SI |
|---|---|---|---|---|---|
| Oya1 | CPXV | Huh-7 | >0.6 | 0.22 ± 0.01 | >2.71 ± 0.21 |
| Oya1 | CPXV | NR-596 | >0.6 | 0.20 ± 0.01 | >3.01 ± 0.25 |
| Oya2 | CPVX | Huh-7 | >0.6 | 0.24 ± 0.01 | >2.71 ± 0.13 |
| Oya2 | CPVX | NR-596 | >0.6 | 0.20 ± 0.03 | >3.01 ± 0.41 |

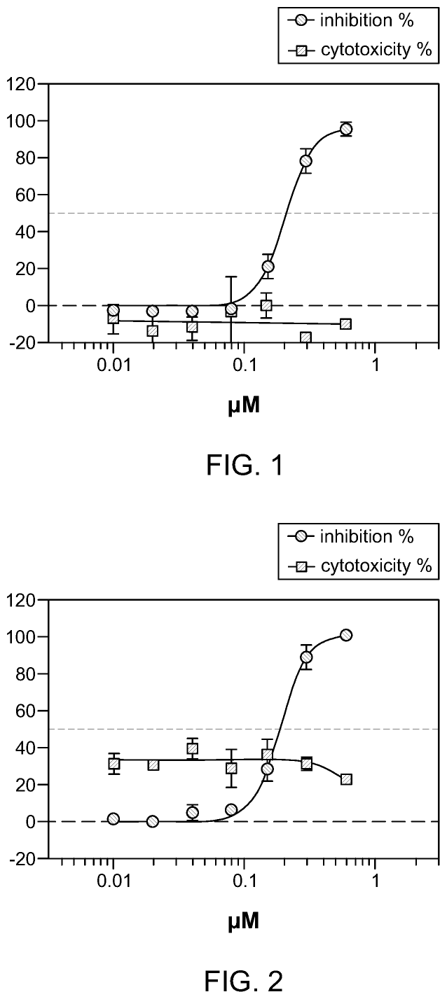

These results are depicted in FIGS. 1-4.

FIG. 1 depicts the antiviral activity of the disclosed compound Oya1 on CPXV virus infected Huh7 cells at a MOI of 0.1 for 24 hours. The percent inhibition that the test compounds displayed is indicated by (●), whereas the percent toxicity is indicated by (▪).

FIG. 2 depicts the antiviral activity of the disclosed compound Oya1 on CPXV virus infected NR596 (VeroE6) cells at a MOI of 0.1 for 24 hours. The percent inhibition that the test compounds displayed is indicated by (●), whereas the percent toxicity is indicated by (▪).

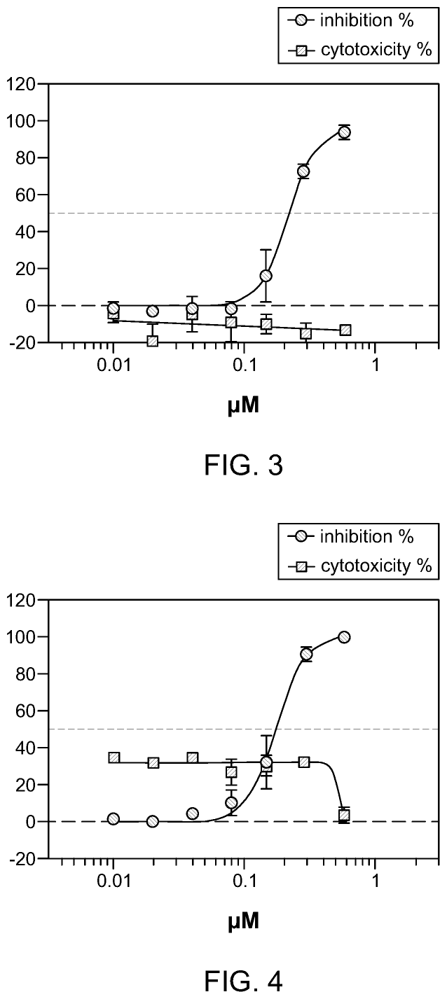

FIG. 3 depicts the antiviral activity of the disclosed compound Oya2 on CPXV virus infected Huh7 cells at a MOI of 0.1 for 24 hours. The percent inhibition that the test compounds displayed is indicated by (●), whereas the percent toxicity is indicated by (▪).

FIG. 4 depicts the antiviral activity of the disclosed compound Oya2 on CPXV virus infected NR596 (VeroE6) cells at a MOI of 0.1 for 24 hours. The percent inhibition that the test compounds displayed is indicated by (●), whereas the percent toxicity is indicated by (▪).

### VACV Assay MOI 0.1

The same procedure as described herein above is used except that VACV is substituted for CPXV.

The results of this experiment is summarized in Table 4.

TABLE 4

| Drug | Virus | Cell type | CC₅₀ | IC₅₀ | SI |
|---|---|---|---|---|---|
| Oya1 | VACV | Huh-7 | >0.6 | 0.05 ± 0.01 | >14.55 ± 3.61 |
| Oya1 | VACV | NR-596 | >0.6 | 0.02 ± 0.01 | >46.81 ± 9.93 |
| Oya2 | VACV | Huh-7 | >0.6 | 0.04 | >15.42 ± 0.25 |
| Oya2 | VACV | NR-596 | >0.6 | 0.01 | >50.65 ± 3.01 |

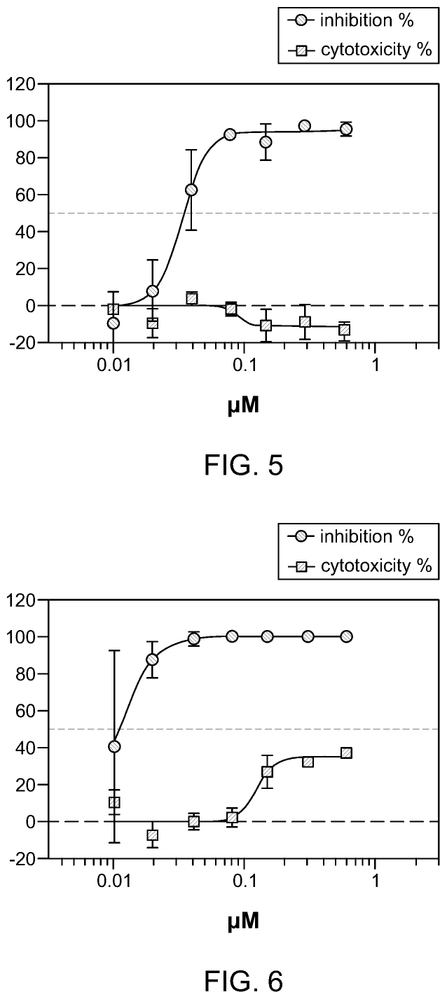

These results are depicted in FIGS. 5-8.

FIG. 5 depicts the antiviral activity of the disclosed compound Oya1 on VACV virus infected Huh7 cells at a MOI of 0.1 for 24 hours. The percent inhibition that the test compounds displayed is indicated by (●), whereas the percent toxicity is indicated by (▪).

FIG. 6 depicts the antiviral activity of the disclosed compound Oya1 on VACV virus infected NR596 (VeroE6) cells at a MOI of 0.1 for 24 hours. The percent inhibition that the test compounds displayed is indicated by (●), whereas the percent toxicity is indicated by (▪).

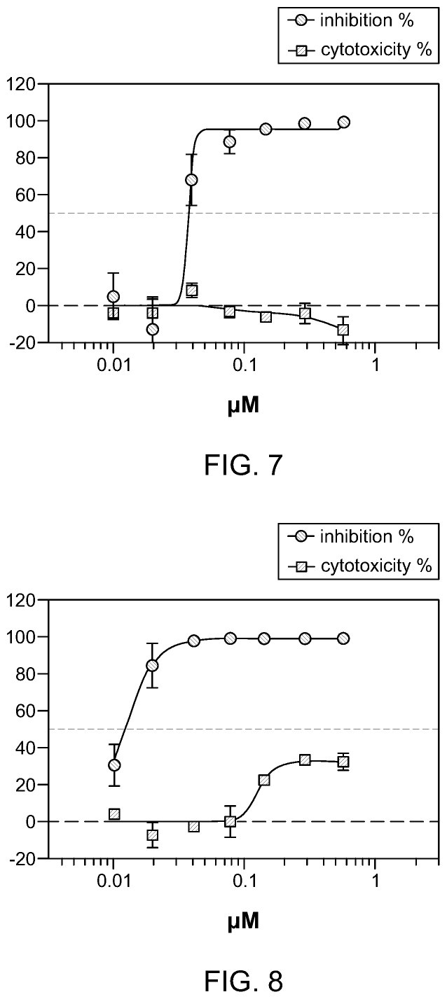

FIG. 7 depicts the antiviral activity of the disclosed compound Oya2 on VACV virus infected Huh7 cells at a MOI of 0.1 for 24 hours. The percent inhibition that the test compounds displayed is indicated by (●), whereas the percent toxicity is indicated by (▪).

FIG. 8 depicts the antiviral activity of the disclosed compound Oya2 on VACV virus infected NR596 (VeroE6) cells at a MOI of 0.1 for 24 hours. The percent inhibition that the test compounds displayed is indicated by (●), whereas the percent toxicity is indicated by (▪).

### Kits

The present disclosure further relates to kits for use by medical or other trained personnel, as well as for use by trained subjects for delivery of the disclosed compositions to a subject. In general, the disclosed kits comprise:
A) an aqueous composition as described herein containing from about 0.1 mg/kg to about 5 mg/kg of the subject's body mass of the subject to which the disclose Poxviridae virus antiviral compound is to be administered; and
B) a means for delivering the composition to a subject.

The compositions of the disclosed kits can comprise the following concentrations of the disclosed compound: 0.5 mg/mL, 1 mg/mL, 1.5 mg/ml, 2 mg/ml, 2.5 mg/mL, 3 mg/mL, 3.5 mg/ml, 4 mg/mL, 4.5 mg/mL 5 mg/mL, 5.5 mg/mL, 6 mg/mL, 7 mg/mL, 7.5 mg/mL, 8 mg/mL, 8.5 mg/mL, 9 mg/mL, 9.5 mg/mL, 10 mg/mL of Oya1 and/or Oya2.

The disclosed compositions can be delivered by any means in keeping with standard pharmaceutical or medical practice. The disclosed aqueous compositions can be administered in any manner chosen by the formulator. Non-limiting examples include parenteral delivery, i.e., intravenous, subcutaneous, and intramuscular. As used herein, “means for delivery” and “delivery device” are used interchangeably. Means for delivery include, but are not limited to, syringes, needles, infusion pumps, injectors. Syringes and injectors can be, for example, single-dose, multi-dose, fixed-dose or variable-dose. Examples of injectors include, but are not limited to, pen injectors, auto-injectors, and electronic patch injector systems. One convenient means for delivering the disclosed compositions is by single use disposable auto injectors. One non-limiting example is a single use injector configured like the single injector sold under the Tradename MOLLY™. Non-limiting examples of injectors are described in U.S. Pat. Nos. 7,442,185; 8,038,649; 8,062,255; 8,075,517; 8,235,952; 8,277,412; 8,529,510; and 8,551,054.

The kits can comprise any suitable means for delivery. In some embodiments the means for delivery provides for the adjustment of delivery volume. For example, the kit may comprise a delivery device that is capable of holding a single dose volume of 0.75 mL is capable of delivering 3 mg/mL of compound when the concentration of the compound is 4 mg/mL. As such, the formulator can provide delivery devices having a higher concentration of compound and adjust the delivered volume to provide an amount of compound that is less than the amount in the entire solution. In another embodiment the kit comprises a delivery device that contains a sufficient amount of a composition to allow for administration of multiple doses from the delivery device.

The following are non-limiting examples of compositions that can comprise the disclosed kits.

One example is a kit comprising:

A) an aqueous composition containing:
a) 5 mg/mL of Oya1; and
b) the balance a carrier system, comprising:
   i) a tonicity agent; and
   ii) water
   wherein the tonicity agent is present in an amount such that the concentration in the final composition is from about 1% to about 5% weight to volume and the carrier system is present in an amount such that the concentration of the disclosed compound has a concentration of 2 mg/mL; and

B) a means for delivering the aqueous composition.

A further aspect of the disclosure relates to kits which comprise a solid composition for reconstitution. The amount of compound in the container of dry composition can be in any convenient amount. For example, a container comprising 5 mg of a disclosed Poxviridae virus antiviral agent can have a demarcation line indicating a final volume of 1 mL. The user can then reconstitute the composition by adding sufficient carrier to create a composition comprising 4 mg/mL of the compound. The formulator also has options for use according to the instructions. For example, the instructions can direct the user to withdraw a sufficient amount according to the prescribed dose. If the prescribed dose is 3 mg/mL the user will withdraw 0.75 mL's of the 4 mg/mL solution for delivery to the subject. Therefore, instructions for re-constitution can afford the user with the proper method of reconstitution, as well as the amount of re-constituted formula to be delivered to a subject.

A set of instructions can be included in any of the herein described kits. The instructions can relate to the dosing amount, timing of dosing, and reconstitution of the composition when the kit contains a dry composition, methods of disposal of delivery means and unused composition, and the like.

### Antiviral Disinfecting Compostions

The disclosed antiviral disinfecting compositions can be fully formulated, i.e., an aqueous based-solution ready for use, or the disclosed compositions can comprise separate components that are combined by the consumer at the time of use. For example, as disclosed herein, the Poxviridae virus antiviral agents and adjunct materials can be in a dry form that is admixed with water and other carriers at the time of use. Alternatively, the compositions can be impregnated or otherwise disposed upon a substrate and when ready for application to a situs, can be re-constituted by the addition of water.

In one aspect, the disclosed compositions relate to aqueous solutions comprising;
a) one or more of the disclosed Poxviridae virus inhibitors;
b) one or more disinfecting agents; and
c) a carrier;
wherein the pH of the composition is from about 3 to about 8.

One embodiment of this aspect relates to compositions comprising:
a) one or more of the disclosed Poxviridae virus inhibitors;
b) hydrogen peroxide; and
c) a carrier;
wherein the pH of the composition is from about 3 to about 8.

A general example of this embodiment includes compositions comprising:
a) from about 2 mM to about 100 mM of one or more of the Poxviridae virus inhibitors;
b) from about 0.01% by weight (3 mM) to about 30% by weight (8.8×10³ mM) of hydrogen peroxide; and
c) the balance a carrier;
wherein the pH of the composition is from about 3 to about 8.

Another embodiment of this aspect relates to compositions comprising:
a) one or more of the disclosed Poxviridae virus inhibitors;
b) hydrogen peroxide;
c) a buffer system; and
d) a carrier;
wherein the pH of the composition is from about 3 to about 8.

A general example, of this embodiment includes compositions comprising:
a) from about 2 mM to about 100 mM of one or more of the disclosed Poxviridae virus inhibitors;
b) from about 0.01% by weight (3 mM) to about 30% by weight (8.8×10³ mM) of hydrogen peroxide;
c) from about 0.01% to about 50% by weight of a buffer system; and
d) the balance a carrier;
wherein the pH of the composition is from about 3 to about 8.

A further embodiment of this aspect relates to compositions comprising:
a) one or more of the disclosed Poxviridae virus inhibitors;
b) hydrogen peroxide;
c) a stabilizer system; and
d) a carrier;
wherein the pH of the composition is from about 3 to about 8.

A general example, of this embodiment includes compositions comprising:
a) from about 2 mM to about 100 mM of one or more of the disclosed Poxviridae virus inhibitors;
b) from about 0.01% by weight (3 mM) to about 30% by weight (8.8×10³ mM) of hydrogen peroxide;
c) from about 0.01% to about 50% by weight of a stabilizer system; and
d) the balance a carrier;
wherein the pH of the composition is from about 3 to about 8.

A yet further embodiment of this aspect relates to compositions comprising:
a) one or more of the disclosed Poxviridae virus inhibitors;
b) hydrogen peroxide;
c) a buffer system;
d) a stabilizer system; and
e) a carrier;
wherein the pH of the composition is from about 3 to about 8.

A general example, of this embodiment includes compositions comprising:
a) from about 2 mM to about 100 mM of one or more of the disclosed Poxviridae virus inhibitors;
b) from about 0.01% by weight (3 mM) to about 30% by weight (8.8×10³ mM) of hydrogen peroxide;
c) from about 0.01% to about 50% by weight of a buffer system;
d) from about 0.01% to about 50% by weight of a stabilizer system; and
e) the balance a carrier;
wherein the pH of the composition is from about 3 to about 8.

In another aspect, the disclosed compositions relate to aqueous solutions comprising;
a) one or more of the disclosed Poxviridae virus inhibitors;
b) one or more disinfecting agents; and
c) a carrier;
wherein the pH of the composition is from about 3 to about 8.

One embodiment of this aspect relates to compositions comprising:
a) one or more of the disclosed Poxviridae virus inhibitors;
b) one or more peroxy acids; and
c) a carrier;
wherein the pH of the composition is from about 3 to about 8.

A general example of this embodiment includes compositions comprising:
a) from about 2 mM to about 100 mM of one or more of the Poxviridae virus inhibitors;
b) from about 0.01% by weight (3 mM) to about 30% by weight (8.8×10³ mM) of one or more peroxy acids; and
c) the balance a carrier;
wherein the pH of the composition is from about 3 to about 8.

Another embodiment of this aspect relates to compositions comprising:
a) one or more of the disclosed Poxviridae virus inhibitors;
b) one or more peroxy acids;
c) a buffer system; and
d) a carrier;
wherein the pH of the composition is from about 3 to about 8.

A general example, of this embodiment includes compositions comprising:
a) from about 2 mM to about 100 mM of one or more of the disclosed coronavirus inhibitors;
b) from about 0.01% by weight (3 mM) to about 30% by weight (8.8×10³ mM) of one or more peroxy acids;
c) from about 0.01% to about 50% by weight of a buffer system; and
d) the balance a carrier;
wherein the pH of the composition is from about 3 to about 8.

A further embodiment of this aspect relates to compositions comprising:
a) one or more of the disclosed Poxviridae virus inhibitors;
b) one or more peroxy acids;
c) a stabilizer system; and
d) a carrier;
wherein the pH of the composition is from about 3 to about 8.

A general example, of this embodiment includes compositions comprising:
a) from about 2 mM to about 100 mM of one or more of the disclosed Poxviridae virus inhibitors;
b) from about 0.01% by weight (3 mM) to about 30% by weight (8.8×10³ mM) of one or more peroxy acids;
c) from about 0.01% to about 50% by weight of a stabilizer system; and
d) the balance a carrier;
wherein the pH of the composition is from about 3 to about 8.

A yet further embodiment of this aspect relates to compositions comprising:
a) one or more of the disclosed Poxviridae virus inhibitors;
b) one or more peroxy acids;
c) a buffer system;
d) a stabilizer system; and
e) a carrier;
wherein the pH of the composition is from about 3 to about 8.

A general example, of this embodiment includes compositions comprising:
a) from about 2 mM to about 100 mM of one or more of the disclosed Poxviridae virus inhibitors;
b) from about 0.01% by weight (3 mM) to about 30% by weight (8.8×10³ mM) of one or more peroxy acids;
c) from about 0.01% to about 50% by weight of a buffer system;
d) from about 0.01% to about 50% by weight of a stabilizer system; and
e) the balance a carrier;
wherein the pH of the composition is from about 3 to about 8.

In a further aspect, the disclosed compositions relate to aqueous solutions comprising;
a) one or more of the disclosed Poxviridae virus inhibitors;
b) one or more surfactants; and
c) a carrier;
wherein the pH of the composition is from about 3 to about 8.

One embodiment of this aspect relates to compositions comprising:
a) one or more of the disclosed Poxviridae virus inhibitors;
b) a surfactant system wherein the surfactant is chosen from one or more anionic surfactants, one or more zwitterionic (amphoteric) surfactants. One or more non-ionic surfactants, one or more cationic surfactants, or mixtures thereof; and
c) a carrier.

The disclosed antiviral disinfecting compositions can comprise a surfactant system, comprising:
i) optionally from about 25% to about 60% by weight of, on or more anionic surfactants;
ii) optionally from about 15% to about 45% by weight of one or more zwitterionic (amphoteric) surfactants;
iii) optionally from about 0.5% to about 10% by weight of one or more nonionic surfactants; or
iv) optionally from about 5% to about 15% by weight of one or more cationic surfactants.

In as still further aspect of the disclosed antiviral compositions, comprise:
a) one or more of the disclosed Poxviridae virus antiviral compounds;
b) one or more quaternary ammonium salts; and
c) the balance carriers and adjunct ingredients.

In one embodiment of this aspect, the disclosed antiviral disinfecting compositions comprise:
a) one or more of the disclosed Poxviridae virus antiviral compounds;
b) one or more quaternary ammonium salts; and
c) one or more dispersing agents;
d) trichloromelamine; and
e) the balance carriers and adjunct ingredients.

In example of this aspect, the following solid composition is dissolved in water to deliver a liquid antiviral disinfecting composition.
a) from about 0.5% to about 5% by weight of one or more of the disclosed Poxviridae virus antiviral compounds;
b) from about 10% to about 90% by weight of one or more quaternary ammonium salts;
c) from about 5% to about 60% by weight of one or more dispersing agents; and d) from about 5% to about 30% by weight of trichloromelamine.

### Hydrogen Peroxide

The disclosed compositions can comprise hydrogen peroxide as the source of hydrogen peroxide in any concentration from about 0.0017% by weight (0.5 mM) to about 30% by weight (8.8×10³ mM). In one embodiment, the hydrogen peroxide concentration is from about 0.5% by weight to about 5% by weight. In another embodiment, the hydrogen peroxide concentration is from about 0.1% by weight to about 1% by weight. In a yet further embodiment, the hydrogen peroxide concentration is from about 0.1% by weight to about 4% by weight.

In another embodiment of the disclosed compositions, the solid component described herein can be provided in a container or other suitable package and the user can purchase a medical source of hydrogen peroxide, for example, a 3% solution of stabilized hydrogen peroxide from a store or pharmacy and admix an amount of the purchased hydrogen peroxide with the solid component as directed by the directions listed on a kit or on a package containing the disclosed solid component.

### Peroxyacids

The disclosed antimicrobial compositions can comprise from about 0.01 weight % to about 50 weight % of one or more peroxyacids. In one iteration, the disclosed compositions can comprise from about 0.05 weight % to 5 weight % of one or more peroxyacids. In another iteration, the disclosed compositions can comprise from about 0.05 weight % to 5 weight % of one or more peroxyacids. In a further iteration, the disclosed compositions can comprise from about 0.5 weight % to 10 weight % of one or more peroxyacids. In a yet another iteration, the disclosed compositions can comprise from about 1 weight % to 5 weight % of one or more peroxyacids. In a yet further iteration, the disclosed compositions can comprise from about 0.5 weight % to 2 weight % of one or more peroxyacids. In a still further iteration, the disclosed compositions can comprise from about 5 weight % to 25 weight % of one or more peroxyacids.

When the disclosed compositions comprise a two component system wherein the two components are combined prior to use, the component comprising the peroxy acid can comprise from about 0.01% to about 100% by weight of one or more peroxy acids. In one embodiment wherein the first component is a solid comprising one or more of the disclosed a-keto acids, the first component can comprised from about 0.01% to about 99.99% by weight of one or more carboxylic acids that can form a peroxy acid upon addition of a source of hydrogen peroxide by the user. In one embodiment, wherein the solid component comprises a buffer system, the first component can comprise from about 0.01% to about 90% by weight of a carboxylic acid that can form a peroxy acid upon addition of a source of hydrogen peroxide by the user.

The one or more peroxyacids can be purchased or the peroxyacids can be formed from the corresponding carboxylic acids. In one embodiment, the peroxyacid or combination of peroxyacids are be formed by combining a hydrogen peroxide (H₂O₂) solution with the desired amount of a carboxylic acid or carboxylic acid blend. In the case of higher molecular weight fatty acids, a solvent as part of the carrier can be required to fully solubilize the fatty acid. The H₂O₂ solution also can be added to previously made peroxyacids such as peroxyacetic acid, peroxyglutaric acid or various peroxy fatty acids to produce the peroxyacid composition admixture. In one iteration, the compositions can comprise from about 1 weight % to about 50 weight % of free hydrogen peroxide. In another iteration, the compositions can comprise from about, 5 weight % to about 25 weight % of hydrogen peroxide.

Suitable C₁-C₁₈ peroxyacids are peroxyfatty acids, monoperoxy- or diperoxydicarboxylic acids, and peroxy aromatic acids. The C₂-C₁₈ peroxyacids employed in the present invention may be structurally represented as follows:

R¹⁰⁰CO₃H

wherein R¹⁰⁰ is a hydrocarbon moiety having from about 1 to 17 carbon atoms (a C₈ peroxyacid is generally represented structurally as C₇CO₃H). R¹⁰⁰ can be substituted in the chain, for example, —OH, —CO₂H, or the chain can comprise heteroatoms as in the case of alkyether carboxylic acids. R¹⁰⁰ can be saturated or unsaturated, linear, branched or cyclic alkyl.

Non-limiting examples of suitable C₂-C₁₈ carboxylic fatty acids which can be reacted with hydrogen peroxide to form peroxyfatty acids include such saturated fatty acids as acetic (C₂), propionic (C₃), butyric (C₄), pentanoic (C₅), hexanoic (C₆), heptanoic (C₇), octanoic (C₈), nonanoic (C₉), decanoic (C₁₀), undecanoic (C₁₁), dodecanoic (C₁₂), tridecanoic (C₁₃), tetradecanoic (C₁₄), hexadecanoic (C₁₆), and octadecanoic (C₁₈). These acids can be derived from both natural and synthetic sources. Natural sources include animal and vegetable fats or oils which should be fully hydrogenated. Synthetic acids can be produced by the oxidation of petroleum wax.

Other suitable acids are the C₆-C₁₈ peroxyacids derived from the oxidation of dicarboxylic acids and aromatic acids. Suitable dicarboxylic acids include adipic acid (C₆) and sebacic acid (C₁₀). Examples of a suitable aromatic acid include benzoic acid, phthalic acid, terephthalic acid, hydroxy benzoic acid, etc. These acids can be reacted with hydrogen peroxide to form the peracid form suitable for use in the disclosed compositions. Non-limiting examples include monoperoxy- or diperoxyadipic acid, monoperoxy- or diperoxysebacic acid, and peroxybenzoic acid.

### Surfactant

The disclosed compositions can comprise from about 0.05% to about 0.2% by weight of a surfactant. In further aspect, the disclosed compositions can comprise from about 0.05% to about 0.2% by weight of a non-ionic surfactant. In one embodiment the surfactant has an HLB of from about 10 to about 20. One aspect of the disclosed compositions comprises a surfactant having an HLB of from about 12 to about 18. A further aspect of the disclosed compositions comprises a surfactant having an HLB of from about 13 to about 16. Another embodiment of the disclosed compositions comprise from about 0.1% to about 0.2% by weight of a surfactant.

In one embodiment, the compositions comprise a nonionic surfactant having an HLB of from about 10 to about 20. One aspect of the disclosed compositions comprises a nonionic surfactant having an HLB of from about 12 to about 18. A further aspect of the disclosed compositions comprises a nonionic surfactant having an HLB of from about 13 to about 16. Another embodiment of the disclosed compositions comprise from about 0.1% to about 0.2% by weight of a nonionic surfactant.

Suitable surfactants include anionic surfactants, for example, linear alkyl sulfates. Non-limiting examples of linear alkyl sulfate surfactants include C₁₀ (decyl) sulfate, C₁₂ (dodecyl) sulfate, and C₁₄ (tetradecyl) sulfate. In addition, mixtures of two or more alkyl surfactants can be used. Suitable salts of linear alkyl sulfates include ammonium, sodium, and potassium.

In addition, branched alkyl surfactants can be used in the disclosed compositions, for example, mid-chain branched alkyl sulfate surfactants as disclosed in U.S. Pat. No. 6,232,282 included herein by reference in its entirety.

Suitable nonionic surfactants for use in the disclosed compositions include polyoxyethylene C₆-C₁₂ alkylphenyl ethers, polyoxyethylene sorbitan tri(C₁₂-C₁₈)-alkanoates, polyoxyethylene sorbitan di(C₁₂-C₁₈)-alkanoates, polyoxyethylene sorbitan mono-, di-, and tri-(C₁₂-C₁₈)-alkanoates, and polyoxyethylene C₁₂-C₂₀ alkyl ethers.

One category of suitable nonionic surfactants for use in the disclosed compositions are the polyoxyethylene C₆-C₁₂ alkylphenyl ethers having the formula:

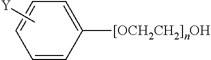

wherein Y is a C₆-C₁₂ alkyl unit and n is an index from 5 to 40. Non-limiting examples of C₆-C₁₂ alkylphenyl ethers includes polyoxyethylene(5) isooctylphenyl ethers sold under the tradenames IGEPAL™ CA-520 and IGEPAL™ CO-520, polyoxyethylene(8) isooctylphenyl ethers sold under the tradename TRITON™ X-114, polyoxyethylene(9) nonylphenyl ether sold under the tradename IGEPAL™ CO-630, polyoxyethylene(10) isooctylphenyl ether sold under the tradename TRITON™ X-100, polyoxyethylene(branched) nonylphenyl ethers sold under the tradename TRITON™ N-101, polyoxyethylene(12) nonylphenyl ether sold under the tradename IGEPAL™ CO-720, polyoxyethylene(12) isooctylphenyl ether sold under the tradename IGEPAL™ CA-720, polyoxyethylene(40) nonylphenyl ether sold under the tradename IGEPAL™ CO-890, and polyoxyethylene(40) isooctylphenyl ether sold under the tradename TRITON™ X-405.

Another category of nonionic surfactants for use in the disclosed compositions are polyoxyethylene sorbitan mono-, di-, and tri-(C₁₂-C₁₈)-alkanoates, non-limiting examples of which include polyoxyethylene(20) sorbitan trioleate sold under the tradename TWEEN™ 85, polyoxyethylene(20) sorbitan monooleate sold under the tradename TWEEN™ 80, polyoxy-ethylene(20) sorbitan monostearate sold under the tradename TWEEN™ 60, polyoxyethyl-ene(20) sorbitan monopalmitate sold under the tradename TWEEN™ 40, and polyoxyethyl-ene(20) sorbitan monolaurate sold under the tradename TWEEN™ 20.

A further category of nonionic surfactants for use in the disclosed compositions are polyoxyethylene C₉-C₂₀ alkyl ethers, non-limiting examples of which include ethoxylate alcohols having the formula:

RO(CH₂CH₂O)ₘH

wherein R is a linear or branched alkyl group having from 6 to 20 carbon atoms and m is an integer of about 2 to about 20. On example of suitable ethoxylate alcohol surfactants are the NEODOL™ ethoxylated alcohols from Shell Chemicals. Non-limiting examples of suitable ethoxylated alcohols include NEODOL™ 91-5, NEODOL™ 91-6, NEODOL™ 91-8, NEODOL™ 91-9, NEODOL™ 23-6.5, NEODOL™ 25-5, NEODOL™ 25-7, NEODOL™ 25-9, NEODOL™ 25-12, NEODOL™ 45-7, and NEODOL™ 135-7, available from BASF.

### Quaternary Ammonium Salts

The disclosed compositions comprise from about 10% to about 90% by weight of one or more quaternary ammonium salts. In one embodiment the compositions comprise from about 10% to about 90% by weight of any single quaternary ammonium salt.

In another embodiment the compositions comprise from about 20% to about 80% by weight of one or more quaternary ammonium salts. In a further embodiment the compositions comprise from about 20% to about 70% by weight of one or more quaternary ammonium salts. In another further embodiment the compositions comprise from about 20% to about 80% by weight of one or more quaternary ammonium salts. In yet further embodiment the compositions comprise from about 10% to about 60% by weight of one or more quaternary ammonium salts. In a still yet further embodiment the compositions comprise from about 30% to about 60% by weight of one or more quaternary ammonium salts. In a yet another embodiment the compositions comprise from about 40% to about 80% by weight of one or more quaternary ammonium salts. In a still another embodiment the compositions comprise from about 30% to about 70% by weight of one or more quaternary ammonium salts. In a yet still further embodiment the compositions comprise from about 30% to about 40% by weight of one or more quaternary ammonium salts. The disclosed compositions can comprise 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% 40%, 4%1%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 88%, 82%, 83%, 84%, 85%, 86%, 87%, 88%, 89%, or 90% of one or more quaternary ammonium salts by weight of the composition.

One category of quaternary ammonium compounds relates to C₁₀-C₂₀ linear alkyl di-C₁-C₄ linear alkyl benzyl ammonium salts having the formula:

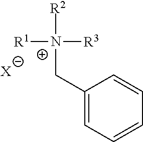

wherein R¹ is C₁₀-C₂₀ linear alkyl, R² and R³ are each independently C₁-C₄ linear alkyl, X is fluorine, chlorine or bromine. In one embodiment X is chlorine. The following are non-limiting examples of this category of quaternary ammonium compounds: decanyl dimethyl benzyl ammonium chloride, undecanyl dimethyl benzyl ammonium chloride, dodecanyl dimethyl benzyl ammonium chloride, tridecanyl dimethyl benzyl ammonium chloride, tetradecanyl dimethyl benzyl ammonium chloride, pentadecanyl dimethyl benzyl ammonium chloride, hexadecanyl dimethyl benzyl ammonium chloride, heptadecanyl dimethyl benzyl ammonium chloride, octadecanyl dimethyl benzyl ammonium chloride, nonadecanyl dimethyl benzyl ammonium chloride, and eicosanyl dimethyl benzyl ammonium chloride.

In one embodiment of this category the quaternary ammonium compounds include: dodecanyl dimethyl benzyl ammonium chloride, tetradecanyl dimethyl benzyl ammonium chloride, hexadecanyl dimethyl benzyl ammonium chloride, and octadecanyl dimethyl benzyl ammonium chloride. The composition can comprise any number of compounds according to this category.

Another category of quaternary ammonium salts relates to C₁₀-C₂₀ linear alkyl di-C₁-C₄ linear alkyl mono-substituted benzyl ammonium salt having the formula:

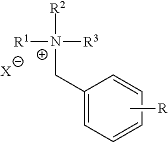

wherein R is from a C₁-C₄ linear alkyl substitution, R¹ is C₁₀-C₂₀ linear alkyl, R² and R³ are each independently C₁-C₄ linear alkyl, X is fluorine, chlorine or bromine. In one embodiment X is chlorine.

The following are non-limiting examples of this category of quaternary ammonium compounds: decanyl dimethyl ethylbenzyl ammonium chloride, undecanyl dimethyl ethylbenzyl ammonium chloride, dodecanyl dimethyl ethylbenzyl ammonium chloride, tridecanyl dimethyl ethylbenzyl ammonium chloride, tetradecanyl dimethyl ethylbenzyl ammonium chloride, pentadecanyl dimethyl ethylbenzyl ammonium chloride, hexadecanyl dimethyl ethylbenzyl ammonium chloride, heptadecanyl dimethyl ethylbenzyl ammonium chloride, octadecanyl dimethyl ethylbenzyl ammonium chloride, nonadecanyl dimethyl ethylbenzyl ammonium chloride, and eicosanyl dimethyl ethylbenzyl ammonium chloride.

In one embodiment of this category the quaternary ammonium compounds include: dodecanyl dimethyl ethylbenzyl ammonium chloride, tetradecanyl dimethyl ethylbenzyl ammonium chloride, hexadecanyl dimethyl ethylbenzyl ammonium chloride, and octadecanyl dimethyl ethylbenzyl ammonium chloride. The composition can comprise any number of compounds according to this category. In a further embodiment the compositions comprise dodecanyl dimethyl ethylbenzyl ammonium chloride and tetradecanyl dimethyl ethylbenzyl ammonium chloride

Another category of quaternary ammonium salts relates to N—C₁-C₂₀ linear alkyl substituted or unsubstituted pyridinium salt having the formula:

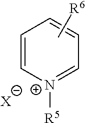

wherein R⁶ is from 0 to 3 independently chosen C₁-C₄ linear alkyl substitutions, R⁵ is C₁-C₂₀ linear alkyl, X is fluorine, chlorine or bromine. In one embodiment X is chlorine.

The following are non-limiting examples of pyridinium salts according to the present disclosure: N-dodecyl pyridinium chloride, N-tetradecyl pyridinium chloride, N-hexadecyl pyridinium chloride, N-octadecyl pyridinium chloride and N-eicosanyl pyridinium chloride (cetyl pyridium chloride). In one embodiment the pyridinium salt is cetyl pyridinium chloride.

### Buffer System

The disclosed compositions have a pH of from about 3 to about 8. In one embodiment the pH is from about 5 to about 7. In another embodiment, the pH is from about 5 to about 6. In a further embodiment, the pH is from about 4.5 to about 5.5. In a further embodiment, the pH is about 5. In a still further embodiment, the pH is about 6. The compositions, however, can have any pH from about 3 to about 8 or any fractional part thereof, for example, a pH of 3, 3.5, 4, 4.5, 5, 5.5, 6, 6.5, 7, 7.5, and 8.

The disclosed compositions can comprise a buffer system to maintain the pH of the compositions whether pre-formulated as a liquid, diluted at the time of use, or whether constituted at the time of use, at a pH of from about 3 to about 8. In one embodiment the pH is from about 5 to about 7. In another embodiment, the pH is from about 5 to about 6. In a further embodiment, the pH is from about 4.5 to about 5.5. In a further embodiment, the pH is about 5. In a still further embodiment, the pH is about 6. The compositions, however, can comprise a buffer system to buffer the pH from about 3 to about 8 or any fractional part thereof, for example, a pH of 3, 3.5, 4, 4.5, 5, 5.5, 6, 6.5, 7, 7.5, and 8.

The formulator, depending upon the level of antimicrobial activity desired, can adjust the pH of the solution to be compatible with the type of microorganism being treated or the situs of application, for example, the skin of a burn victim, an open wound, an inert surface, or a food surface.

### Carrier

The disclosed compositions can comprise a liquid carrier when not in the solid form. The user can add a liquid carrier to a dry or solid formulation to complete the composition, for example, the user in one embodiment will add an amount of water to a powder or other solid formulation. In another embodiment, the user can be directed by the instructions of a kit to add an amount of hydrogen peroxide, for example, a 3% by weight solution of hydrogen peroxide. More than one carrier can be added or more than one carrier can comprise the liquid embodiments disclosed herein.

In one embodiment, water is the carrier. In another embodiment, the carrier can be an aqueous solution of a source of hydrogen peroxide, for example, an aqueous solution of hydrogen peroxide or an aqueous solution of a source of hydrogen peroxide, i.e., a perborate. In addition, C₁-C₁₀ linear, branched, and cyclic aliphatic alcohols can be either carriers alone or can be a part of the carrier system. In one embodiment, methanol is added as a co-carrier.

Non-limiting examples of suitable organic acid buffer systems include acetic acid/sodium acetate, glycolic acid/sodium glycolate, lactic acid/sodium lactate, succinic acid/mono sodium succinate, adipic acid/mono sodium adipate, malic acid/mono sodium malate, tartaric acid/mono sodium tartrate, and the like. Non-limiting examples of suitable inorganic buffer systems include phosphate buffer systems.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A method for treating a subject infected with a Poxviridae virus, comprising: administering to a subject a therapeutically effective amount of 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula:

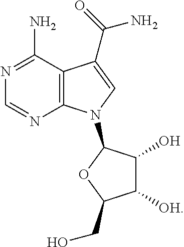 [SMILES: NC(=O)c1cn([C@@H]2O[C@H](CO)[C@@H](O)[C@H]2O)c2ncnc(N)c12]

2. A method for treating a subject infected with a Poxviridae virus, comprising: administering to a subject a therapeutically effective amount of 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula:

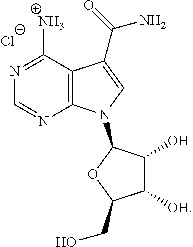 [SMILES: [Cl-] ; NC(=O)c1cn([C@@H]2O[C@H](CO)[C@@H](O)[C@H]2O)c2ncnc([NH3+])c12]

3. The method according to either claim 1 or 2, wherein the effective amount is from about 0.1 mg/kg to about 5 mg/kg of the body mass of the subject.

4. The method according to either claim 1 or 2, wherein the effective amount is selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 5.0 mg/kg of the body mass of the subject.

5. A method for inhibiting the RNA-dependent RNA polymerase of the Poxviridae virus, comprising administering to a subject having a Poxviridae virus infection a therapeutically effective amount of:
i) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula: 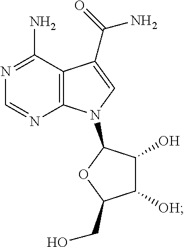 [SMILES: NC(=O)c1cn([C@@H]2O[C@H](CO)[C@@H](O)[C@H]2O)c2ncnc(N)c12]
ii) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula: 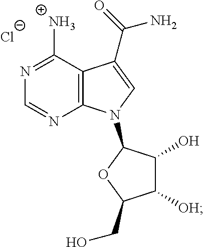 [SMILES: [Cl-] ; NC(=O)c1cn([C@@H]2O[C@H](CO)[C@@H](O)[C@H]2O)c2ncnc([NH3+])c12]
or
iii) mixtures thereof.

6. The method according to claim 5, wherein the effective amount is from about 0.1 mg/kg to about 5 mg/kg of the body mass of the subject.

7. The method according to claim 5, wherein the effective amount is selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 5.0 mg/kg of the body mass of the subject.

8. A stop-gap method for preventing the spread of Poxviridae virus comprising administering to a subject having a Poxviridae virus infection a therapeutically effective amount of:
i) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula: 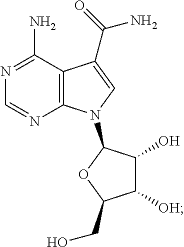 [SMILES: NC(=O)c1cn([C@@H]2O[C@H](CO)[C@@H](O)[C@H]2O)c2ncnc(N)c12]
ii) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula: 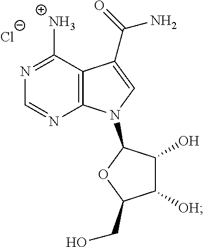 [SMILES: [Cl-] ; NC(=O)c1cn([C@@H]2O[C@H](CO)[C@@H](O)[C@H]2O)c2ncnc([NH3+])c12]
or
iii) mixtures thereof.

9. The method according to claim 8, wherein the effective amount is from about 0.1 mg/kg to about 5 mg/kg of the body mass of the subject.

10. The method according to claim 8, wherein the effective amount is selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 5.0 mg/kg of the body mass of the subject.

11. A method for preventing the emergence of a drug-resistant strain of a Poxviridae virus, comprising administering to a subject having a Poxviridae virus infection a therapeutically effective amount of:
i) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula: 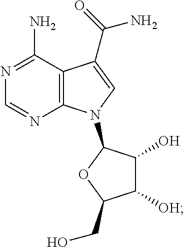 [SMILES: NC(=O)c1cn([C@@H]2O[C@H](CO)[C@@H](O)[C@H]2O)c2ncnc(N)c12]
ii) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula: 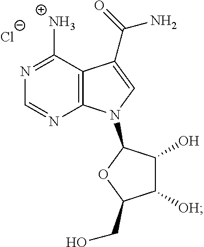 [SMILES: [Cl-] ; NC(=O)c1cn([C@@H]2O[C@H](CO)[C@@H](O)[C@H]2O)c2ncnc([NH3+])c12]
or
iii) mixtures thereof.

12. The method according to claim 11, wherein the effective amount is from about 0.1 mg/kg to about 5 mg/kg of the body mass of the subject.

13. The method according to claim 11, wherein the effective amount is selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 5.0 mg/kg of the body mass of the subject.

14. A method of prophylactically treating a subject uninfected with an Poxviridae virus comprising administering to an uninfected subject reasonably suspected as having been exposed, of currently being exposed, or in the future of being exposed to an Poxviridae virus a therapeutically effective amount of:
i) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide having the formula: 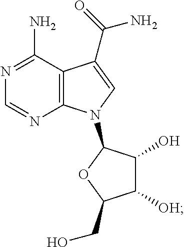 [SMILES: NC(=O)c1cn([C@@H]2O[C@H](CO)[C@@H](O)[C@H]2O)c2ncnc(N)c12]
ii) 4-amino-7-((2R,3R,4S,5R)-3,4-dihydroxy-5-(hydroxymethyl)tetrahydrofuran-2-yl)-7H-pyrrolo[2,3-d]pyrimidine-5-carboxamide hydrochloride having the formula: 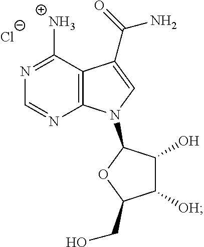 [SMILES: [Cl-] ; NC(=O)c1cn([C@@H]2O[C@H](CO)[C@@H](O)[C@H]2O)c2ncnc([NH3+])c12]
or
iii) mixtures thereof.

15. The method according to claim 14, wherein the effective amount is from about 0.1 mg/kg to about 5 mg/kg of the body mass of the subject.

16. The method according to claim 14, wherein the effective amount is selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 5.0 mg/kg of the body mass of the subject.

17. A pharmaceutical composition comprising:
a) a therapeutically effective amount of:
i) 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide having the formula: 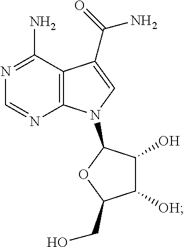 [SMILES: NC(=O)c1cn([C@@H]2O[C@H](CO)[C@@H](O)[C@H]2O)c2ncnc(N)c12]
ii) 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide hydrochloride having the formula: 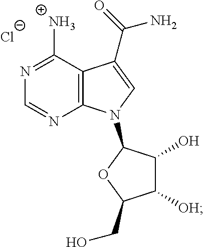 [SMILES: [Cl-] ; NC(=O)c1cn([C@@H]2O[C@H](CO)[C@@H](O)[C@H]2O)c2ncnc([NH3+])c12]
or
iii) mixtures thereof; and
b) the balance a pharmaceutically acceptable carrier.

18. The composition according to claim 17, comprising from about 10 mg to about 250 mg of 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide and/or the hydrochloride salt thereof.

19. The composition according to either claim 17 or 18, wherein the composition is in the form of an oral-use composition.

20. The composition according to either claim 17 or 18, wherein the composition is in the form of a pill.

21. The composition according to either claim 17 or 18, wherein the composition is in the form of a capsule.

22. The composition according to either claim 17 or 18, wherein the composition is in the form of a nasal delivery composition.

23. The composition according to either claim 17 or 18, wherein the composition is in the form of a sterile injectable composition.

24. Use of a pharmaceutical composition according to either claim 17 or 18 for the treatment of Poxviridae virus in combination with a vaccine, a therapeutic, and/or other small molecule drug intended for treatment of Coronavirus as a combination therapy to treat Poxviridae virus in a subject.

25. The use according to claim 24, wherein the combination therapy is effective to inhibit and/or prevent the emergence of drug resistant strains of Poxviridae virus.

26. A kit comprising a pharmaceutical composition, the composition comprising:
a) one or more Poxviridae virus inhibitors chosen from:
i) 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide having the formula: 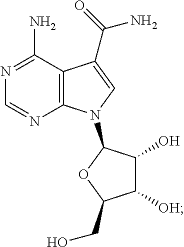 [SMILES: NC(=O)c1cn([C@@H]2O[C@H](CO)[C@@H](O)[C@H]2O)c2ncnc(N)c12]
ii) 4-amino-7-((2R,3R,4S,5R)-3,4-dihydroxy-5-(hydroxymethyl)tetrahydro-furan-2-yl)-7H-pyrrolo[2,3-d]pyrimidine-5-carboxamide hydrochloride having the formula, 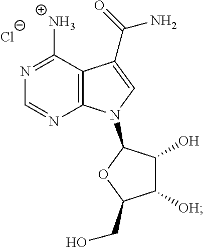 [SMILES: [Cl-] ; NC(=O)c1cn([C@@H]2O[C@H](CO)[C@@H](O)[C@H]2O)c2ncnc([NH3+])c12]
or
iii) mixtures thereof; and
b) optionally the balance a pharmaceutically acceptable carrier.
